(19) United States
(12) Patent Application Publication
(10) Pub. No.: US 2018/0249959 A1
(43) Pub. Date: Sep. 6, 2018

(54) MAPPING CATHETER

(71) Applicant: Peter Osypka Stiftung, Grenzach-Wyhlen (DE)

(72) Inventor: Peter Osypka, Grenzach-Wyhlen (DE)

(21) Appl. No.: 15/907,924

(22) Filed: Feb. 28, 2018

(30) Foreign Application Priority Data
Mar. 1, 2017 (DE) 10 2017 001971
Feb. 8, 2018 (EP) 18020052.9

(51) Int. Cl. A61B 5/00; A61B 5/042

(52) U.S. Cl. CPC A61B 5/6859; A61B 18/1492; A61B 5/0422

(57) ABSTRACT

The invention is directed to a mapping catheter, more particularly to a high resolution mapping catheter for mapping the atrial endocardium in high resolution in order to provide information on the propagation of the electrical signals to treat atrial fibrillation. The catheter is mainly configured for mapping, but—if required—may also be used for ablation, for stimulation and for cardioversion.

The invention relates to a mapping catheter comprising a catheter shaft having a proximal section and a distal section and at least one lumen and
an electrode array being self-expandable from a collapsed structure to an expanded structure and being arranged at the distal section of the catheter shaft when expanded;
the electrode array comprises a plurality of multi-pole coils being grouped around the central longitudinal axis of the electrode array, whereby each multi-pole coil has an electrical supply section and a sensing section, whereby the sensing section of each coil comprises a plurality of poles being arranged adjacent to each other, each coil has a lumen through which a pre-shaped wire made of shape memory metal runs; the pre-shaped wire has a distal and a proximal end; and whereby the expanded electrode array is shaped according to the pre-shaped wires so that in the use position the poles have close contact to the endocardial tissue.

### FIELD OF THE INVENTION

The invention is directed to a mapping catheter, more particularly to a high resolution mapping catheter for mapping the atrial endocardium in high resolution in order to provide information on the propagation of the electrical signals to treat atrial fibrillation. The catheter is mainly configured for mapping, but—if required—may also be used for ablation, for stimulation and for cardioversion.

### BACKGROUND

Atrial fibrillation occurs if rapid, disorganized electrical signals cause the atria to contract very fast and irregularly. As a result blood is not pumped completely into the ventricles and the atria and the ventricles do not work together as they should. Some blood remains in the atria so that blood clots may be formed causing a stroke or an embolus.

The risk of atrial fibrillation increases with the age and affects millions of people, and the number is rising.

Atrial fibrillation may be treated using HF ablation to restore a normal heart rhythm. For this procedure, the electrode catheter is inserted into a major vein and is guided into the atria.

Research on the propagation of the electrical signals in the heart is ongoing and more information for understanding the electrical problems in the heart chambers is required.

There is thus still a need to provide a device for high resolution mapping of the atrial endocardium (the inner layer of tissue that lines the heart chambers) to determine the location of arrhythmia origin. Furthermore the device should be suitable for subsequently applied individual therapies. Therefore the device should be configured for ablation, stimulation or cardioversion.

### SUMMARY OF THE INVENTION

The disclosure relates to a mapping catheter comprising an electrode array which consists of a plurality of multi-pole coils having high pole density and allowing a good contact to the endocardial tissue. Inside the coils there are wires of shape memory material allowing an individual pre-shaping of the multipole coils. The pre-shaping is based on medical imaging data like MRT data or CT data.

The invention relates to a mapping catheter comprising a catheter shaft having a proximal section and a distal section and at least one lumen and

an electrode array being self-expandable from a collapsed structure to an expanded structure and being arranged at the distal section of the catheter shaft when expanded;
the electrode array comprises a plurality of multi-pole coils being grouped around the central longitudinal axis of the electrode array, whereby each multi-pole coil has an electrical supply section and a sensing section, whereby the sensing section of each coil comprises a plurality of poles being arranged adjacent to each other, each coil has a lumen through which a pre-shaped wire made of shape memory metal runs; the pre-shaped wire has a distal and a proximal end; and whereby the expanded electrode array is shaped according to the pre-shaped wires so that in the use position the poles have close contact to the endocardial tissue.

The electrode array can be used for mapping, for stimulation and for ablation in order to treat atrial fibrillation. The array can further be used for cardioversion.

Shape memory material is e.g. nitinol. The term “nitinol” shall mean all common shape memory materials.

The sensing section is the part of the coil having poles. The length of the sensing section can vary e.g. in the range of 5 to 20 mm. A suitable length may be 15 mm. The length of the sensing section may be equal for all the coils of the electrode array or may vary.

The term “a plurality of poles being arranged adjacent to each other” means e.g. 16 poles which may be arranged on one coil if 16 wires are wounded to a coil. The poles being arranged adjacent to each other form a “sensing unit”. When using 16 wires with a diameter of 0.12 mm for making the coil the resulting “sensing unit” has a length of 16×0.12 mm, circa 2 mm. A sensing unit of 5-20 mm may be reached by changing the pitch of the coil. The distance between each pole may be adjusted by changing the pitch of the coil.

The term “adjacent to each other” means a small distance between each pole. The distance is less than 5 mm; less than 2 mm or less than 1 mm; e.g. 0.2 mm or less than 0.2 mm; e.g. 0.08 to 0.2 mm.

More than 16 poles per coil may be provided by improving the current coiling technique whereby the maximum number of wires which can be coiled is 16 wires. More than 16 poles per coil may be provided by making combined coils. Combined coils are made by fitting coils into each other. For fitting the coils into each other each coil used must have an increasing diameter. When fitting two coils into each other, a combined coil having 32 poles results. When fitting three coils into each other, a combined coil having 48 poles results. It is also possible to combine coils having single poles with blank coils. By combining coils it is possible to use the mapping catheter not only for mapping but for ablation and cardioversion too.

The term “a plurality of coils” means more than 2 coils, e.g. 4 coils or more than 4 coils; e.g. 4 to 16 coils.

The total number of poles in the array depends on the number of sensing units on each coil and on the number of coils. Both numbers may vary, so that a high flexibility and high variability is given with regard to the pole arrangement and the total number of poles.

A mapping catheter with 128 poles can be made by arranging 8 coils, each coil having 16 poles. A mapping catheter with 192 poles can be made by arranging 12 coils, each coil having 16 poles.

All the poles have a close contact to the endocardial tissue of the atrium or to the tissue of the pulmonary vein. With one mapping procedure a large number of electrical signals might be simultaneously received. Thus, the distribution of electrical signals can be recorded (exact mapping). It is possible to create an individual map of electrical signals for each patient. The perfect contact of the multipole coils to the endocardial surface is given due to the pre-shaped nitinol wires running inside the coil.

In one embodiment the mapping catheter is suitable to record electrical signals in the pulmonary vein and in the region of the pulmonary vein ostia. The multi-pole coils are grouped around a longitudinal axis. Due to the pre-shaped nitinol wires running inside the coils it is guaranteed that the coils and thus the poles fit closely to the inner tissue of the pulmonary veins.

In one embodiment the mapping catheter is suitable to record electrical signals in the left or right atrium. The multipole coils are grouped around a longitudinal axis. Preferably a guide wire runs along the central longitudinal axis of the mapping catheter and thus of the electrode array. The multi-pole coils are grouped around the guide wire. Due to the pre-shaped nitinol wires running inside the coils of the electrode array it is guaranteed that the coils and thus the poles fit closely to the endocardial tissue. The pre-shaped nitinol wire is freely movable inside the coil. The diameter of the nitinol wire is in the range of 0.1 to 0.5 mm. The pre-shaped nitinol wire preferably extends beyond the distal end point of the coil.

In one embodiment the pre-shaped nitinol wires running through the coils and extending beyond the distal end point of the coil are distally connected to each other e.g. welded together. This arrangement results in better stability of the electrode array. The coils are thus held in position when the electrode array expands and do not slip away.

The contact of the multipole coils to the endocardial tissue may be improved by using a guidewire, running along the longitudinal axis of the mapping catheter and thus of the electrode array. The nitinol wires running inside the multipole coils and extending beyond the distal end point of the coil are distally attached to the guidewire e.g. by welding. Thus, the nitinol wires are attached to the guidewire and connected to each other via the guidewire. The pressure of the coils against the endocardial tissue and thus the contact of the poles is adjustable by pulling or pressing the guidewire. A close contact between pole and endocardial surface is important.

In one embodiment the guide wire is circle shaped at its distal end and the pre-shaped nitinol wires running through the coils are attached to the circle.

The guidewire is preferably made of nitinol or of stainless steel. Further poles, sensors or a LED light source can be attached to the guidewire.

Adjacent coils grouped around the longitudinal axis and the guide wire, respectively have the same distance and are thus grouped regularly or the distance between adjacent coils differs, thus the coils are irregularly grouped.

The part of the atrium which can be mapped simultaneously is called the “mapping areal”. The areal may be planar, concave or convex. The electrode array is shaped convex by pressing the guidewire. The electrode array is shaped concave by pulling the guidewire.

The multipole coil is made of a plurality of isolated wires running parallel. The poles are made by removing a small part of the isolation layer. The coil must be soft, flexible and yet stable too. Flexibility, softness and stability of the coil may be influenced by the coil material used, the diameter and the pitch of the coil.

The diameter of the wire forming the coil is in the range of 0.08 to 0.20 mm, preferably 0.08 mm; 0.1 mm; 0.12 mm; 0.15 mm; 0.20 mm, said range of the coil diameter guarantees to have a soft and elastic coil. Due to the softness and elasticity the contact to the endocardial tissue is improved.

The diameter of the coil is in the range of 0.4 mm-2.0 mm, preferably 0.6-0.8 mm. The coils used may have an individual diameter. The coils may be combined by fitting single coils into each other resulting is a combined coil having more than 16 poles.

Biocompatible electrically conducting materials may be chosen to form the coils, such as stainless steel, Elgiloy (Co—Cr—Ni Alloy) MP35N (Ni—Co-alloy), Isotan (CuNi44), Nitinol, Platinum, Pt—Ir, tungsten, Cu, Cu—Pt.

The coils are isolated using poleyimide, poleyurethane, silicone, Pebax, PTFE, poleyamide or any biocompatible plastic.

The poles are blank zones on the coils that are not coated with any isolating material. The coil surface may also be covered by gold bumps formed at a pre-determined part of the coil. The gold bumps may have a diameter of 0.1 to 0.2 mm.

Each coil has a plurality of poles arranged in one straight line or in a zig-zag line. Using an arrangement in zig-zag line a close as well as a safe arrangement of the poles is possible.

The number of poles and the number of coil-wires carrying the poles may vary. It is important that the distance between two adjacent poles is small in order to have a high pole density and thus a high mapping density. The zig zag arrangement is advantageous with regard to a small and safe distance of the poles.

Electrical signals of the atrium are recorded in order to determine the location of arrhythmia origin. Due to the high pole density of the above described array, it is possible to map an area of the atrium in high resolution. When a misguided signal is located, said position can be mapped again with even higher resolution. The great variation in the pole arrangement helps to map in higher and higher resolution.

The inventive mapping catheter may be used in a computer controlled or an ultrasound controlled mapping process.

If the origin of atrial fibrillation is located, ablation can be done using the same device.

An embodiment being suitable for mapping and ablation comprises a coil where a bare wire is drawn between and parallel to the isolated wire. RF current pulses are transmitted via said bare wire. The material used for said bare wire may be the same material as used for the coils.

The poles allow further stimulation an even cardioversion. Stimulation is done by transmitting an electrical impulse to the poles of the coil.

Cardioversion is done using the above described ablation arrangement. The bare wire is the indifferent pole in the cardioversion process. Bare coils can be arranged between the multipole coils in order to increase the pole area.

The aim of the present invention is the high pole density and the improved contact of the poles to the atrial endocardium due to the pre-shaped wires running inside the coils and due to the guidewire with which the pre-shaped wires are connected. The pre-shaping is based on medical imaging data like MRT, CT, X-ray, ultrasound and the like.

The catheter shaft must have at least one lumen to insert the electrode array.

In one embodiment the catheter shaft has more than one lumen, e.g. two lumens. The second lumen is used to insert a counter pressure device being positioned opposite to the electrode array. The counter pressure device may improve the contact of the poles to the atrial endocardium. A suitable counter pressure device may be a balloon or a nitinol braiding, a screw coil or a spacer.

The inventive electrode array is preferably used for mapping the right or left atrium.

Inserting the device into the left atrium is done via the atrial septum e.g. by using a device according to EP2674189 whereby the electrode pole is shaped like an occlusion element being self-expandable and forming a double disc which bridges the left and right atrium.

### REFERENCE SIGNS

1 catheter shaft
2 electrode array
3 multipole coils
4 nitinol wire running inside the multipole coils
5 electrical supply section
6 guide wire having a circle shaped distal end
7 sensing section of the multi pole coil
8 mapping areal
9 guide wire having a straight distal end
10 pole
11 blank wire
12 nitinol braiding used as counter pressure device
13 balloon used as counter pressure device
14 balloon used as counter pressure device
15 spacer used as counter pressure device
16 screw coil used as counter pressure device
17 Steerable catheter
20 pulmonary vein
21 Pulmonary vein ostium
22 atrial septum
30 delivery device

### DESCRIPTION OF THE DRAWINGS

The following figures explain the invention in more detail.

FIG. 1 shows the inventive multi electrode array in side view (FIG. 1a, 1c) and in top view (FIG. 1b, 1b′, 1d, 1e, 1f)

FIG. 1a shows the catheter shaft (1) and the electrode array (2), which is arranged at the distal section of the catheter shaft. The electrode array is expanded. The array consists of multipole coils (3) which are arranged distally to the catheter shaft. The multipole coils (3) are grouped around a guide wire (6). The multipole coils have different sectors, an electrical supply section (5) and a sensing section (7) carrying the poles. The guide wire (6) runs inside the shaft along a longitudinal axis in the center of the electrode array. At its distal end the guide wire is straight or circle shaped. The multipole coils have a lumen. Through said lumen pre-shaped nitinol wires are running. The sensing section (7) has close contact to the endocardium in the use position due to said pre-shaped nitinol wires.

FIG. 1b is a top view along the line A-A of FIG. 1a. The multipole coils (3) are arranged in a circle and have sensing sections (7). The pre-shaped nitinol wires (4) running through the coils extend distally beyond the end of the coils and are connected to the guide wire (9). In the top view the central guide wire is visible as dot. FIG. 1 b shows 6 coils. The distance of each pair of two neighboring coils is varying. Circle (6) shows the circle shaped guide wire and suggests a further embodiment whereby the coils are connected to the circle shaped guide wire as shown in FIG. 1b′.

FIG. 1c is a side view corresponding to FIG. 1a. Compared to FIG. 1a more coils are present.

FIG. 1d is a top view along the line A-A of FIG. 1c. The sensing section (7) encompasses an elliptically shaped area. 12 coils are grouped around guide wire (9)

FIG. 1e shows an electrode array where the mapping area (8) is selected individually taking the anatomy of the heart chambers into account (custom made). The sensing section (7) of each coil has a different length c1, c2, . . . etc.

FIG. 1f shows an array corresponding to FIG. 1b. Compared to FIG. 1b the length of the sensing section (7) is increased due to wavelike deformation of the nitinol wire, running inside the coils. Such an extension of the sensing section allows variation in positioning the poles. It is also possible to have a combination of wavelike shaped coils and of straight coils.

FIG. 2 shows the multi electrode array in side view corresponding to FIG. 1c. Compared to FIG. 1c the sensing section is convex (FIG. 2a) or concave (FIG. 2b). The convex and the concave shape respectively is made by pressing or pulling the guide wire being distally connected to the nitinol wires running through the coils. The sensing are is thus anatomically contoured to fit the atrial area.

FIG. 3 shows the multi electrode array corresponding to FIG. 1e placed in the left atrium. The sensing section (7) and thus the mapping area (8) is shaped according the patient heart chamber anatomy. Individual areas (18) may be mapped in more detail if required. The individual areas are marked by a dashed line. It is also possible to map the ostium of the pulmonary veins (21). Catheter shaft (1) is guided through the atrial septum (22) by means of a delivery device (30), e.g. a delivery device as disclosed in the European publication EP 2674189. The device comprises two nitinol braids having a tube like opening. The device allows access to the left ventricle. The device further allows multisite pacing whereby the nitinol braid functions as indifferent electrode pole and the pole (3) of the coils function as different electrode pole.

FIG. 4 shows the coils in more detail.

FIG. 4a shows a coil being made by coiling 16 insulated wires. Eight electrical poles (10) are made by removing the layer of insulation. The electrical poles are lined up. The coil shown in FIG. 4a is suitable for mapping and for stimulation.

The coil shown in FIG. 4b is suitable for mapping and for stimulation. The electrical poles are offset. (zig zag line). By arranging the poles in zig zag line a close as well as a safe arrangement of poles is given. 16 poles are arranged on one coil.

FIG. 4c shows a coil being suitable for ablation and for cardioversion. Blank wires (11) are arranged between insulated wires carrying the electrical poles (10).

FIG. 4d shows a blank coil which can be positioned between the multipole wires of the electrode array.

FIG. 4e shows a combination of 3 coils; two coils as shown in FIG. 4b and a blank coil. Each coil ha another diameter, so that the single coils may be fitted into each other.

FIG. 5 shows the catheter having a counter pressure device

FIG. 5a shows a catheter shaft (1) having two lumen whereby the second lumen is needed to insert nitinol braid (12) which functions as counter pressure device. The electrode array (2) corresponds to the array shown in FIG. 1a

FIG. 5b shows a balloon (13) being the counter pressure device.

FIG. 5c shows a balloon (13) being the counter pressure device.

FIG. 5d shows a spacer (15) being the counter pressure device.

FIG. 5e shows a screw coil (16) being the counter pressure device.

FIG. 6 is a side view showing schematically the mapping of the pulmonary veins (20)

FIG. 6a shows the catheter shaft (1) and the electrode array (2), which is arranged at the distal section of the catheter shaft. The electrode array is expanded. The array consists of multipole coils (3) which are arranged distally to the catheter shaft. The multi-pole coils are grouped around a longitudinal axis. Due to the pre-shaped nitinol wires (not to be seen in the figure) running inside the coils it is guaranteed that the coils and thus the poles fit closely to the inner tissue of the pulmonary veins (20).

FIG. 6b is a side view showing schematically the mapping of the pulmonary vein ostia (21). Catheter shaft (1) and the electrode array (2), which are arranged at the distal section of the catheter shaft. The electrode array is expanded. The array consists of multipole coils (3) which are arranged distally to the catheter shaft.

FIG. 7 shows the mapping catheter in use position. Steerable catheter (17) is inserted through a vein and guided to the right atrium and further through the septum to the left atrium using device (30)

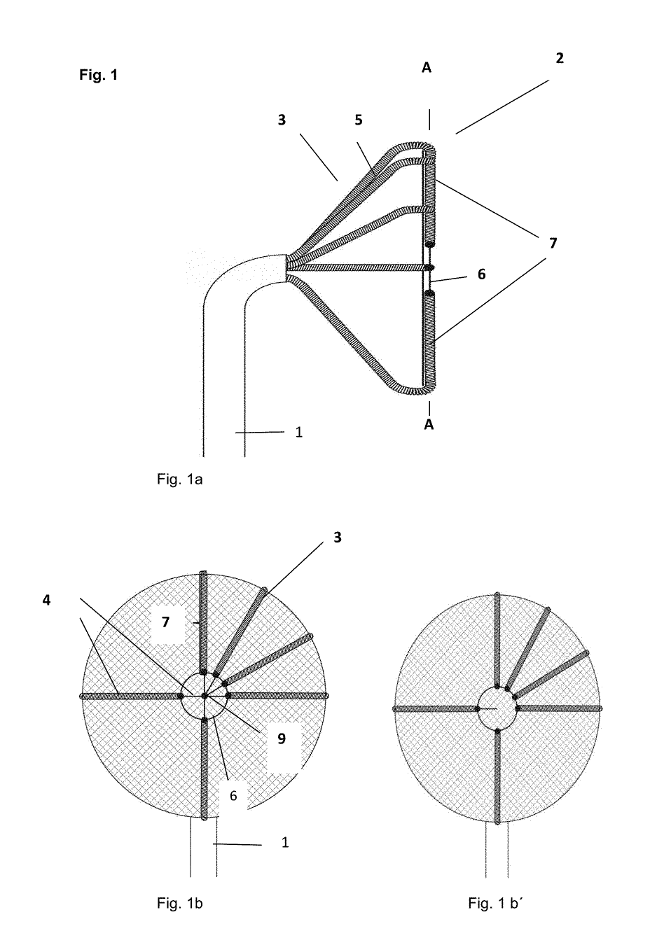

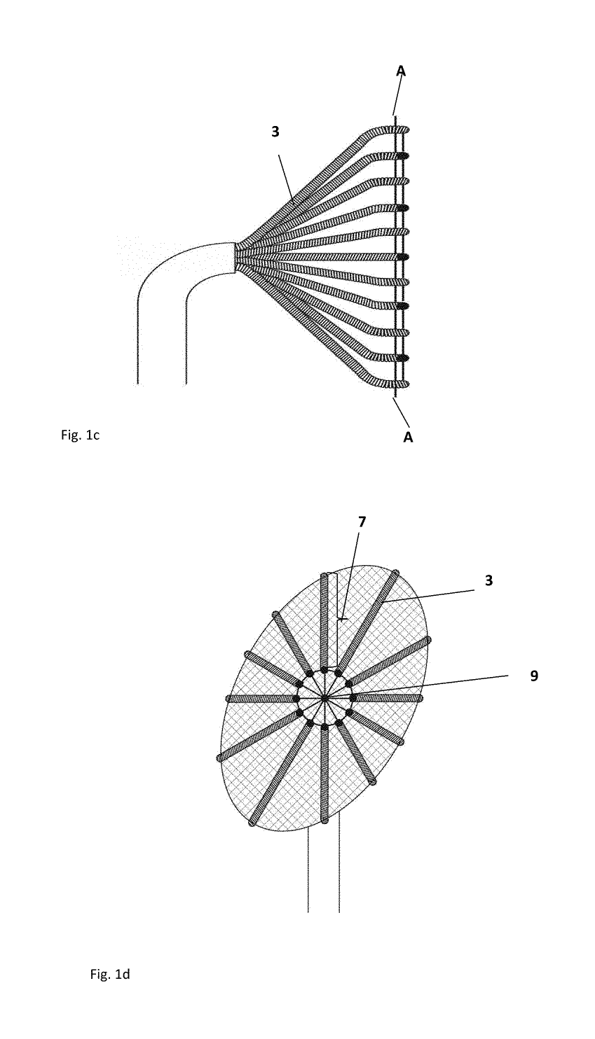

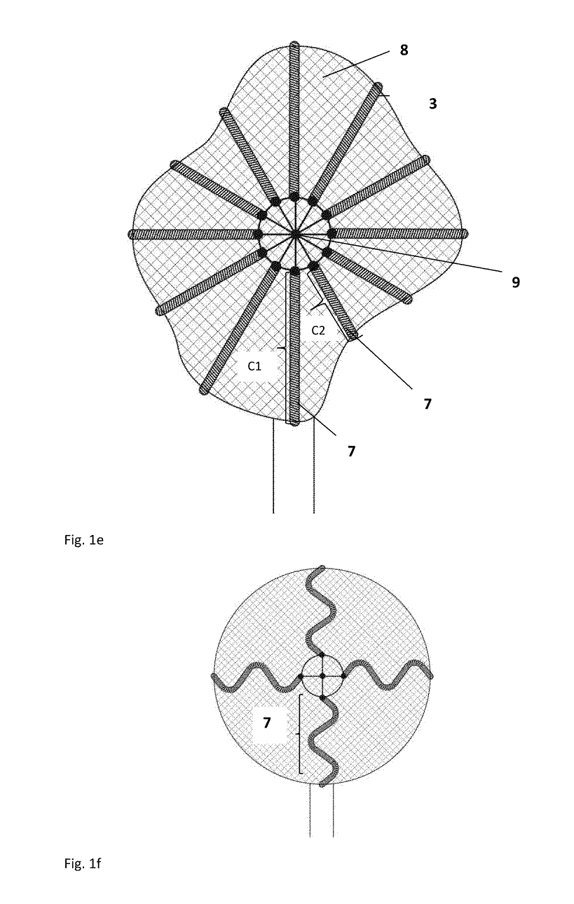

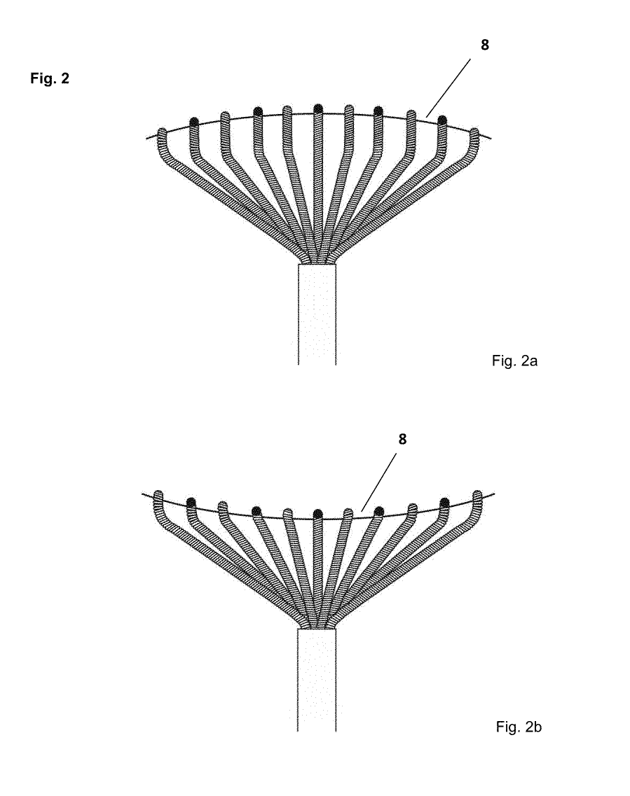

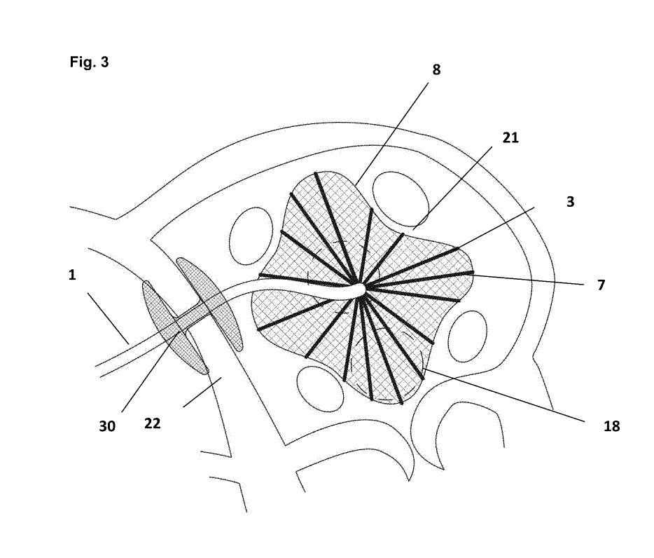

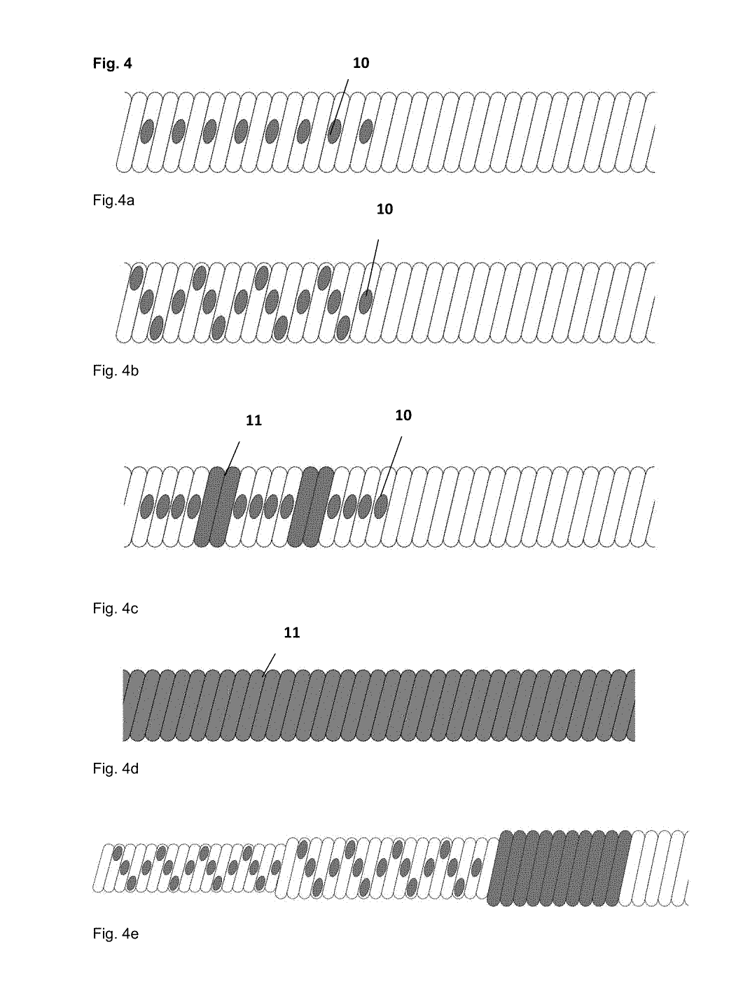

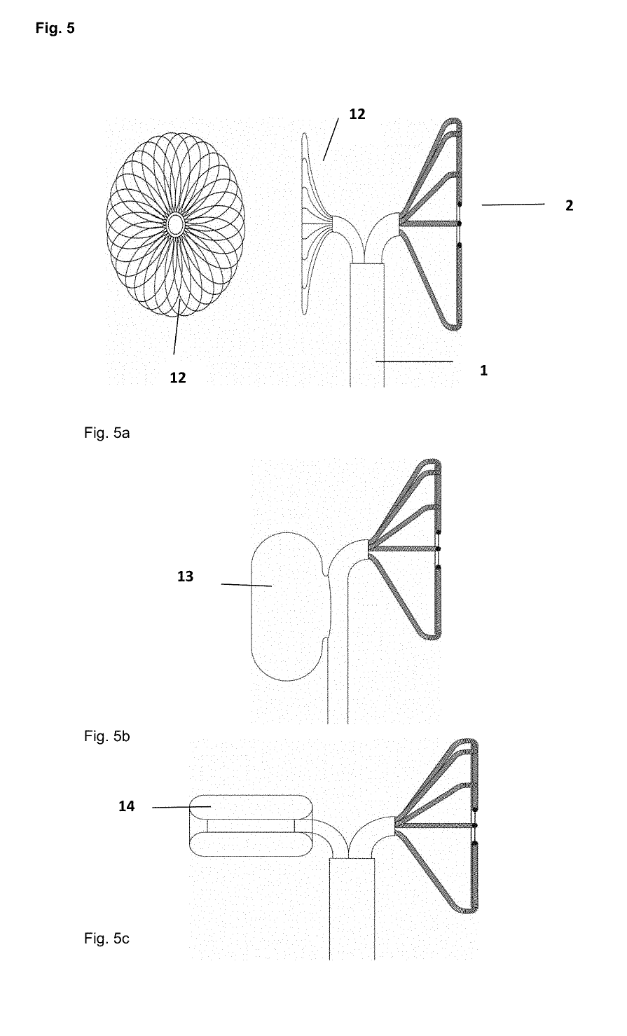

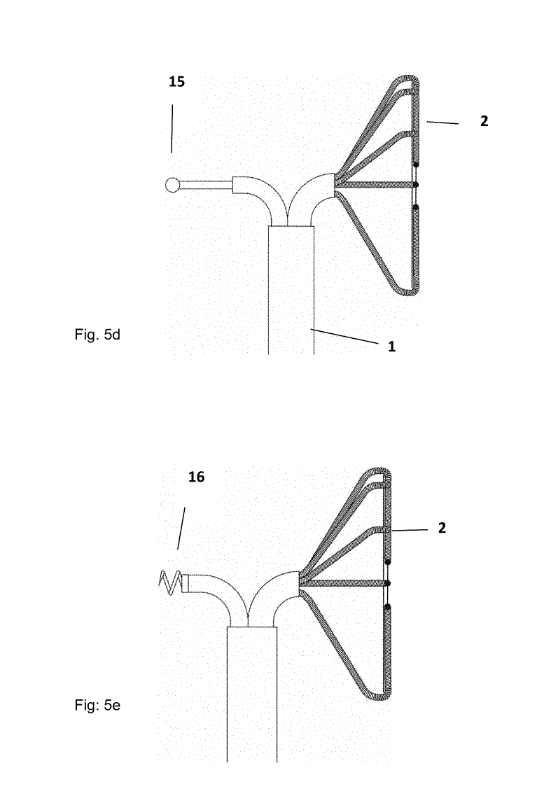

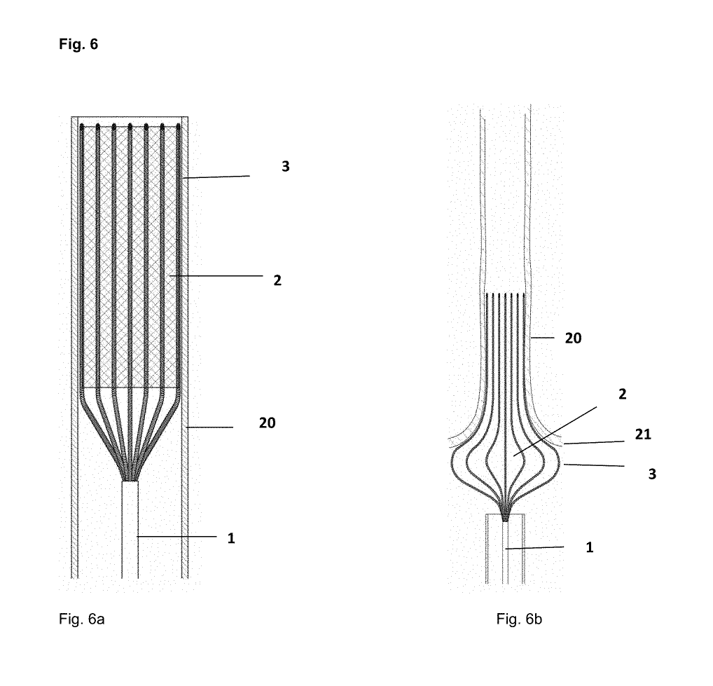

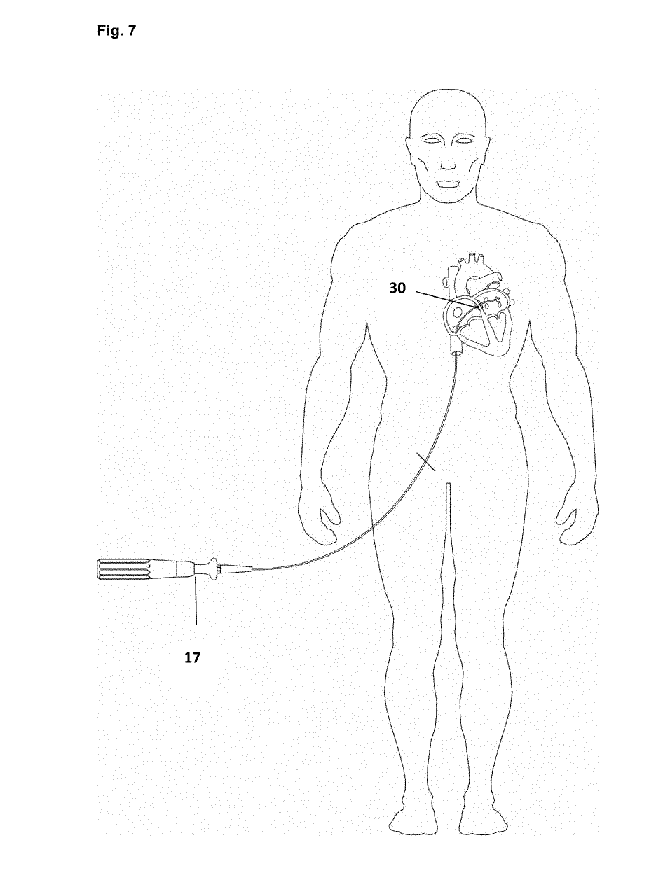

1. A mapping catheter comprising:
a catheter shaft having a proximal section and a distal section and at least one lumen; and
an electrode array being self-expandable from a collapsed structure to an expanded structure and being arranged at the distal section of the catheter shaft when expanded;
wherein the electrode array includes a plurality of multi-pole coils being grouped around a central longitudinal axis of the electrode array, whereby each multi-pole coil has an electrical supply section and a sensing section, whereby the sensing section of each coil includes a plurality of poles being arranged adjacent to each other, wherein each coil has a lumen through which a pre-shaped wire made of shape memory metal runs; and
wherein the pre-shaped wire has a distal end and a proximal end, and whereby the expanded electrode array is shaped according to the pre-shaped wires so that in a use position the poles have close contact to endocardial tissue.

2. The mapping catheter according to claim 1, whereby the shape memory metal is nitinol.

3. The mapping catheter according to claim 1, whereby a guide wire having a circle shaped distal end or a guide wire having a straight distal end runs along the longitudinal axis and the multi pole coils are grouped around the guide wire.

4. The mapping catheter according to claim 3, whereby the pre-shaped nitinol wires running through each multi pole coil extent beyond the distal end point of the coil and whereby each nitinol wire is connected to the distal end of guide wire.

5. The mapping catheter according to claim 3, whereby the pre-shaped nitinol wires running through each multi pole coil are connected to the circle shaped distal end of guide wire.

6. The mapping catheter according to claim 1, whereby the multipole coil is made of a plurality of isolated wires running parallel and the poles are made by removing a small part of the isolation layer.

7. The mapping catheter according to claim 1, having 16 poles per coil.

8. The mapping catheter according to claim 1, whereby two or more coils are combined by being fitted into each other.

9. The mapping catheter according to claim 1, whereby two coils having each 16 poles and a blank coil are combined.

10. The mapping catheter according to claim 1, whereby the diameter of the wire forming the coil is in the range of 0.08 to 0.20 mm.

11. The mapping catheter according to claim 1, having 4-16 multipole coil.

12. The mapping catheter according to claim 1, whereby a LED source is associated with a guide wire.

13. The mapping catheter according to claim 1, whereby an ultrasonic sensor is attached to a guide wire.

14. The mapping catheter according to claim 1, whereby the catheter shaft has two lumens.

15. The mapping catheter according to claim 14, whereby a counter pressure device is inserted through the second lumen.
